(19) United States
(12) Patent Application Publication
(10) Pub. No.: US 2023/0107872 A1
(43) Pub. Date: Apr. 6, 2023

(54) CATHETER TIP WITH INTEGRATED ELECTRONICS PACKAGE AND CATHETER INCORPORATING THE SAME

(71) Applicant: ST. JUDE MEDICAL, CARDIOLOGY DIVISION, INC., St. Paul, MN (US)

(72) Inventors: Fermin Armando LUPOTTI, Lake Forest, CA (US); Scott C. MEYERSON, Ham Lake, MN (US)

(21) Appl. No.: 17/802,368

(22) PCT Filed: Dec. 9, 2020
(86) PCT No.: PCT/US2020/064036

§ 371 Date: Aug. 25, 2022

Related U.S. Application Data
(60) Provisional application No. 62/987,574, filed on Mar. 10, 2020.

(51) Int. Cl. A61B 8/00; A61B 8/12; A61M 25/00

(52) U.S. Cl. CPC A61B 8/4254; A61B 8/12; A61M 25/0012; A61B 2562/0223

(57) ABSTRACT

An intravascular catheter includes a catheter shaft having a distal portion, a plurality of magnetic localization elements disposed within the distal portion, and an integrated electronics package disposed within the distal portion of the catheter shaft. The integrated electronics package, which can be a system on a chip such as an application specific integrated circuit, includes a power supply, a pre-amplifier, a multiplexor, and an imaging element driver. It can also include imaging elements. The magnetic localization elements can include magnetic coils and/or solid state magnetic localization elements, such as anisotropic magnetoresistive sensors, and can also be incorporated into the integrated electronics package.

### CROSS-REFERENCE TO RELATED APPLICATIONS

This application claims the benefit of U.S. provisional application No. 62/987,574, filed 10 Mar. 2020, which is hereby incorporated by reference in its entirety as though fully set forth herein.

### BACKGROUND

The present disclosure relates generally to catheters that are used in the human body. In particular, the present disclosure relates to an intravascular catheter that includes an integrated electronics package in its distal region, such as within its tip.

Catheters are used for an ever-growing number of procedures. For example, catheters are used for diagnostic, therapeutic, and ablative procedures, to name just a few examples. Typically, the catheter is manipulated through the patient's vasculature and to the intended site, for example, a site within the patient's heart.

### BRIEF SUMMARY

Disclosed herein is an intravascular catheter, including: a catheter shaft having a distal portion; a plurality of magnetic localization elements disposed within the distal portion of the catheter shaft; and an integrated electronics package disposed within the distal portion of the catheter shaft. The integrated electronics package includes: a power supply; a pre-amplifier; a multiplexor; and an imaging element driver.

The integrated electronics package can be a system on a chip, such as an application specific integrated circuit.

The plurality of magnetic localization elements can include a plurality of magnetic coils, which can be operably connected to the integrated electronics package. Alternatively or additionally, the plurality of magnetic localization elements can include a plurality of solid state magnetic localization elements, such as a plurality of anisotropic magnetoresistive sensors, and can be mounted within or on the integrated electronics package.

Also disclosed herein is a tip assembly for an intravascular catheter, including: a shell; a plurality of magnetic localization elements disposed within the shell; and an integrated electronics package disposed within the shell. The integrated electronics package includes: a power supply; a pre-amplifier; a multiplexor, and an imaging element driver.

The electronics package can include a system on a chip, such as an application specific integrated circuit.

The plurality of magnetic localization elements can include a plurality of magnetic coils, which can be operably connected to the integrated electronics package. Alternatively or additionally, the plurality of magnetic localization elements can include a plurality of solid state magnetic localization elements, such as a plurality of anisotropic magnetoresistive sensors, which can be mounted within the integrated electronics package.

The instant disclosure also provides a method of manufacturing a tip assembly for an intravascular catheter. The method includes: forming a shell; positioning an integrated electronics package within the shell; positioning a plurality of magnetic localization elements within the shell; and operably connecting the plurality of magnetic localization elements to the integrated electronics package. The integrated electronics package, in turn, includes: a power supply; a pre-amplifier; a multiplexor; and an imaging element driver.

It is contemplated that the plurality of magnetic localization elements can be incorporated into the integrated electronics package. For instance, a plurality of solid state magnetic localization elements can be incorporated into the integrated electronics package.

The integrated electronics package can include an application specific integrated circuit.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of a representative catheter according to aspects of the instant disclosure.

FIG. 2 illustrates a first embodiment of a tip assembly for an intravascular catheter as disclosed herein.

FIG. 3 illustrates a second embodiment of a tip assembly for an intravascular catheter as disclosed herein.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

Aspects of the instant disclosure relate to catheters with electronics packages integrated into their distal ends (e.g., positioned within their distal tip assemblies). Those of ordinary skill in the art will appreciate that the teachings herein can be applied to good advantage in connection with various types of catheters, including, but not limited to, intracardiac echocardiography (ICE) catheters. For instance, the teachings herein can be applied in connection with intravascular catheters as disclosed in U.S. application Ser. No. 15/948,818 (“the '818 application”). As another example, the teachings herein can be applied in connection with catheters such as those disclosed in United States patent application publication no. 2014/0275957 (“the '957 publication”). The '818 application and the '957 publication are hereby incorporated by reference as though fully set forth herein.

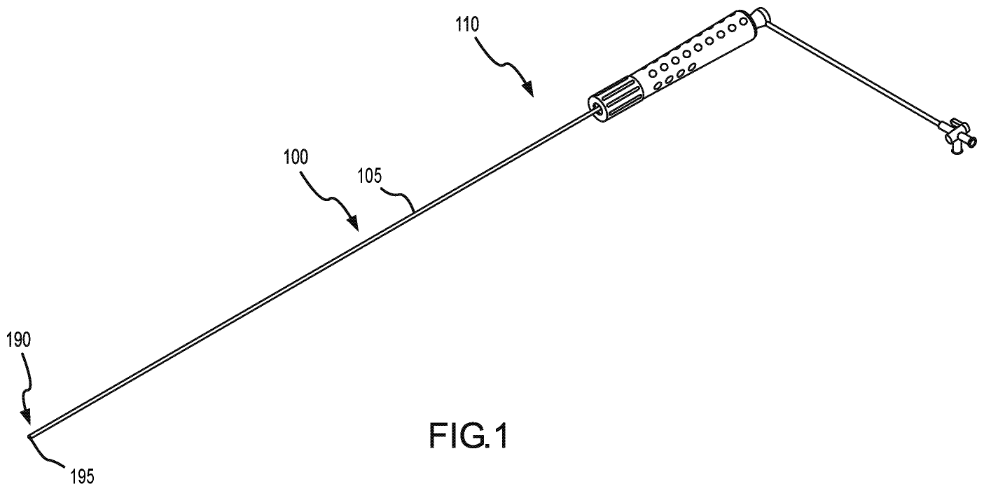

For purposes of illustration, FIG. 1 depicts a perspective view of a representative catheter 100, including a shaft 105 having a proximal portion 110 and a distal portion 190, which terminates in a tip 195. Insofar as the basic construction of catheter 100 will be familiar to those of ordinary skill in the art, the details thereof will be omitted herein, except to the extent relevant to an understanding of the instant disclosure.

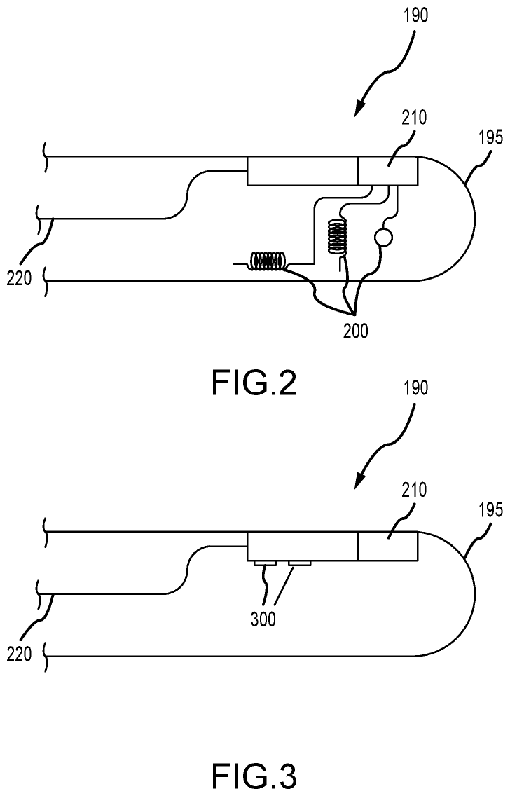

FIG. 2 is a close-up view of distal portion 190, including tip 195, according to a first embodiment of the instant disclosure. A plurality of magnetic localization elements (e.g., magnetic coils 200) are disposed within distal portion 190 (which, for purposes of explanation, can also be referred to as a “shell”). FIG. 2 depicts three orthogonal coils 200. As those of ordinary skill in the art will appreciate, these three orthogonal coils 200 allow tip 195 to be localized with six degrees of freedom within a magnetic localization field generated by an electroanatomical mapping system such as the EnSite Precision™ cardiac mapping system of Abbott Laboratories (Chicago, Ill.).

Also shown within distal portion 190 is an integrated electronics package 210. In embodiments of the disclosure, integrated electronics package 210 is implemented as a system-on-a-chip (SoC), such as an application specific integrated circuit (ASIC) or a field programmable gate array (FPGA).

Integrated electronics package 210 includes a power supply, a pre-amplifier, a multiplexor, and electronics to drive imaging elements, such as transmitters and/or receivers. The imaging elements themselves (e.g., an ultrasonic transducer array, optical fibers, or the like) can also be included within integrated electronics package 210; alternatively, integrated electronics package 210 can include connections to imaging elements that are external to integrated electronics package 210.

Coils 200 are operably connected to integrated electronics package 210, for example to pass power from integrated electronics package 210 to coils 200 and to pass electromagnetic signals (e.g., magnetic field measurements, control signals, and the like) between coils 200 and integrated electronics package 210.

Advantageously, because of the proximity between coils 200 and integrated electronics package 210, coils 200 can be made smaller than extant magnetic localization elements. For instance, extant coils 200 may be from about 0.5 cm to about 1 cm in length with diameters ranging from about 0.3 cm to about 0.5 cm. The proximity between coils 200 and integrated electronics package 210 according to the instant disclosure, however, allows these dimensions to be reduced by about 50%.

Additional cable connectors 220 are provided to connect integrated electronics package 210 (and thus coils 200) to a control unit. Although only one connector 220 is shown in the Figures, those of ordinary skill in the art will appreciate that multiple such connectors 220 can be used and/or bundled through shaft 105.

In another embodiment depicted in FIG. 3, coils 200 are replaced by one or more solid state magnetic localization elements 300, which can be mounted on or within integrated electronics package 210 (e.g., incorporated into the SoC or ASIC). For instance, solid state magnetic localization elements 300 may be anisotropic magnetoresistive (AMR) sensors, such as the AFF811 sensor from Sensitec GmbH (Lahnau, Germany).

In either embodiment (that is, whether the magnetic localization elements are implemented as coils 200 or as solid state elements 300), the proximity between the magnetic localization elements and integrated electronics package 210 advantageously improves the quality of signal communication therebetween (e.g., it offers an improved signal-to-noise ratio relative to extant catheters).

In addition, the burden and expense associated with assembly of catheter 100 is minimized through the incorporation of elements into integrated electronics package 210 (e.g., requiring only a single bundle of connectors 220 (e.g., wires) extending along catheter shaft 105; eliminating additional wiring between the magnetic localization sensors and the integrated electronics package when solid state elements 300 are used).

Although several embodiments have been described above with a certain degree of particularity, those skilled in the art could make numerous alterations to the disclosed embodiments without departing from the spirit or scope of this invention.

For example, integrated electronics package 210 can also include elements that sense the orientation of shaft 105, such as one or more solid state accelerometers.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An intravascular catheter, comprising:
a catheter shaft having a distal portion;
a plurality of magnetic localization elements disposed within the distal portion of the catheter shaft; and
an integrated electronics package disposed within the distal portion of the catheter shaft, wherein the integrated electronics package comprises:
a power supply;
a pre-amplifier;
a multiplexor; and
an imaging element driver.

2. The intravascular catheter according to claim 1, wherein the electronics package comprises a system on a chip.

3. The intravascular catheter according to claim 2, wherein the system on a chip comprises an application specific integrated circuit.

4. The intravascular catheter according to claim 1, wherein the plurality of magnetic localization elements comprises a plurality of magnetic coils.

5. The intravascular catheter according to claim 4, wherein the plurality of magnetic coils are operably connected to the integrated electronics package.

6. The intravascular catheter according to claim 1, wherein the plurality of magnetic localization elements comprises a plurality of solid state magnetic localization elements.

7. The intravascular catheter according to claim 6, wherein the plurality of solid state magnetic localization elements comprises a plurality of anisotropic magnetoresistive sensors.

8. The intravascular catheter according to claim 6, wherein the plurality of solid state magnetic localization elements are mounted within the integrated electronics package.

9. A tip assembly for an intravascular catheter, comprising:
a shell;
a plurality of magnetic localization elements disposed within the shell; and
an integrated electronics package disposed within the shell, wherein the integrated electronics package comprises:
a power supply;
a pre-amplifier;
a multiplexor; and
an imaging element driver.

10. The tip assembly according to claim 9, wherein the electronics package comprises a system on a chip.

11. The tip assembly according to claim 10, wherein the system on a chip comprises an application specific integrated circuit.

12. The tip assembly according to claim 9, wherein the plurality of magnetic localization elements comprises a plurality of magnetic coils.

13. The tip assembly according to claim 12, wherein the plurality of magnetic coils are operably connected to the integrated electronics package.

14. The tip assembly according to claim 9, wherein the plurality of magnetic localization elements comprises a plurality of solid state magnetic localization elements.

15. The tip assembly according to claim 14, wherein the plurality of solid state magnetic localization elements comprises a plurality of anisotropic magnetoresistive sensors.

16. The tip assembly according to claim 14, wherein the plurality of solid state magnetic localization elements are mounted within the integrated electronics package.

17. A method of manufacturing a tip assembly for an intravascular catheter, comprising:
forming a shell;
positioning an integrated electronics package within the shell, wherein the integrated electronics package comprises:
a power supply;
a pre-amplifier;
a multiplexor; and
an imaging element driver;
positioning a plurality of magnetic localization elements within the shell; and
operably connecting the plurality of magnetic localization elements to the integrated electronics package.

18. The method according to claim 17, wherein operably connecting the plurality of magnetic localization elements to the integrated electronics package comprises incorporating the plurality of magnetic localization elements into the integrated electronics package.

19. The method according to claim 18, wherein incorporating the plurality of magnetic localization elements into the integrated electronics package comprises incorporating a plurality of solid state magnetic localization elements into the integrated electronics package.

20. The method according to claim 17, wherein the integrated electronics package comprises an application specific integrated circuit.
